(19) United States
(12) Patent Application Publication
(10) Pub. No.: US 2025/0006308 A1
(43) Pub. Date: Jan. 2, 2025

(54) NANOPORE MEASUREMENT SIGNAL ANALYSIS

(71) Applicant: Oxford Nanopore Technologies PLC, Oxford (GB)

(72) Inventor: Marcus Hudak Stoiber, Cambridge, MA (US)

(73) Assignee: Oxford Nanopore Technologies PLC, Oxford (GB)

(21) Appl. No.: 18/705,183

(22) PCT Filed: Nov. 23, 2022
(86) PCT No.: PCT/GB2022/052965

§ 371 Date: Apr. 26, 2024

Related U.S. Application Data
(60) Provisional application No. 63/283,777, filed on Nov. 29, 2021.

(51) Int. Cl. G16B 40/10; G16B 30/10; G16B 40/20

(52) U.S. Cl. CPC G16B 40/10; G16B 30/10; G16B 40/20

(57) ABSTRACT

A measurement signal measured from a polymer during translocation of the polymer with respect to a nanopore is analysed using an input sequence estimate of the sequence of polymer units of the polymer, and a mapping between the measurement signal and the input sequence estimate. In particular, a sequence slice derived from a slice of the input sequence estimate around a subject polymer unit in the sequence of polymer units, and a signal slice of the measurement signal mapped to the sequence slice by the mapping, are supplied as inputs to a slice machine learning system that provides an output representing an estimate of the identity of the subject polymer unit.

The present invention relates to the analysis of a measurement signal derived from a polymer, for example but without limitation a polynucleotide, during translocation of the polymer with respect to a nanopore.

Measurement systems for estimating a target sequence of polymer units in a polymer using a nanopore wherein the polymer is translocated with respect to the nanopore are known. Some property of the system, for example current through the nanopore, depends on an interaction of the polymer units with the nanopore, and measurements of that property are taken. The property depends on the identity of the polymer units translocating with respect to the nanopore and so the signal over time allows the sequence of polymer units to be estimated. Each polymer unit can be quite small compared to dimensions of the pore, allowing multiple polymer units to affect the signal at a given period of time. Longer range effects may also be present due to interactions of the polymer strand with the nanopore, intra-strand properties like winding or stacking, or interactions between the polymer units and any system used to control their translocation.

The measurement signal needs to be analysed to estimate the underlying polymer units. The accuracy of such analysis is limited by the measurement systems being extremely sensitive. In practice, estimation with high accuracy requires the application of complex algorithms. Such analysis may be performed using machine learning systems, for example neural networks, to provide an output representing an estimate of the identity of polymer units in the polymer, for example nucleotides in the case that the polymer is a polynucleotide.

The present invention is concerned with improving such an analysis to improve the estimation of polymer units.

Some embodiments of the present invention are concerned with detection of modified forms of a canonical polymer unit. In the case of DNA polynucleotides, the canonical nucleotides may be any of the four bases, adenosine, guanosine, cytidine, thymidine, and the modified forms may be nucleotides where a covalent chemical modification is present, for example 5-methyl-cytosine (5mC), 5-hydroxymethyl-cytosine (5hmC), and 6-methyl-adenosine (6 mA).

Chemical modifications to DNA and RNA can affect their function by regulating gene expression and chemical modifications play a vital role in the epigenetic control of gene expression in animal and plants (the way genes are read). Thus there is an important need to be able to determine modifications to both DNA and RNA when sequencing. Due to the chemical nature of many common biological modifications, modified bases are often difficult to detect. As a result, methods have been developed to convert modified bases to aid in their detection. Bisulphite sequencing involves the treatment of DNA with bisulphite to determine methylation and converts canonical cytosine (but not 5mC or 5hmC) to uracil (U) and, as such, canonical cytosine can fairly easily be discriminated from 5mC and 5hmC (but 5mC and 5hmC cannot be discriminated (as disclosed for example in Yu, M., Hon, G. C., Szulwach, K. E., Song. C., Jin, P., Ren. B., He, C. Tet-assisted bisulfite sequencing of 5-hydroxymethylcytosine. Nat. Protocols 2012, 7, 2159). Methods have been developed to discriminate 5mC from 5hmC (as disclosed for example in Liu Y, Siejka-Zielińska P, Velikova G, Bi Y, Yuan F. Tomkova M. Bai C. Chen L, Schuster-Böckler B, Song C X. Bisulfite-free direct detection of 5-methylcytosine and 5-hydroxymethylcytosine at base resolution. Nat Biotechnol. 2019 April; 37 (4):424-429. doi: 10.1038/s41587-019-0041-2. Epub 2019 Feb. 25. PMID: 30804537), but there are no known methods for converting many other common and biologically important modified bases. Furthermore, treatment with bisulphite can lead to degradation of DNA and incomplete desulfonation of pyrimidine residues during the conversion reaction may also lead to difficulties in subsequent amplification of the DNA due to the inhibition of some polymerases. Accordingly, there is desire to be able to detect modifications directly without relying on external data (converted sequence data using bisulphite) or without the need for chemical modifications or other pre-treatment modification steps.

Such modifications alter the measurement signal derived from a polymer during translocation of the polymer with respect to a nanopore, which in principle allows the modified forms of a canonical polymer unit to be detected. However, such detection may in practice be difficult as the alteration of the measurement signal is typically small.

Other embodiments of the present invention are concerned with providing an estimate of the identity of one or more subject polymer units, which allows for detection of errors in a previously derived estimate of the sequence of polymer units and/or for detection of changes from a reference sequence.

According to a first aspect of the present invention, there is provided a method of analysing a measurement signal measured from a polymer during translocation of the polymer with respect to a nanopore, the polymer comprising a sequence of polymer units, the method comprising: deriving an input sequence estimate of the sequence of polymer units, and a mapping between the measurement signal and the input sequence estimate, supplying a sequence slice derived from a slice of the input sequence estimate around a subject polymer unit in the sequence of polymer units, and a signal slice of the measurement signal measurement signal, the sequence slice and signal slice being mapped to each other by the mapping, as inputs to a slice machine learning system that provides an output representing an estimate of the identity of the subject polymer unit.

It has been shown by the present inventors that use of a sequence slice derived from a slice of the input sequence estimate around a subject polymer unit in the sequence of polymer units, and a signal slice of the measurement signal, where the sequence slice and the signal slice are mapped to each other by a mapping between the measurement signal and the input sequence estimate provides estimation of the identity of the subject polymer unit with high accuracy compared to other techniques.

The input sequence estimate may take different forms.

In one form, the input sequence estimate may be an initial estimate of the sequence of polymer units provided as the output of an initial machine learning system that has been supplied with the measurement signal as an input.

In another form, the input sequence estimate may be a reference sequence in respect of the polymer, for example a known reference extracted from a library or a consensus sequence derived from multiple measurement signals derived from a common polymer. In that case, the mapping between the measurement signal and the input sequence estimate, i.e. the reference sequence, may be derived using an initial machine learning system that is supplied with the measurement signal as an input and provides an output that is an initial sequence estimate of the sequence of polymer units. Then, there may be derived both a reference mapping between the reference sequence and the initial sequence estimate, and a signal mapping between the measurement signal and the initial sequence estimate. This allows the desired mapping to be derived from the reference mapping and the signal mapping.

In some types of embodiment, the output may represent an estimate of the identity of the subject polymer unit between categories including canonical polymer unit and at least one modified form of the canonical polymer unit. This allows for detection of modified forms of canonical polymer units with high accuracy.

In other types of embodiment, the output may represent an estimate of the identity of the subject polymer unit between categories including a set of canonical polymer units. This allows for detection of errors in a previously derived estimate of the sequence of polymer units and/or for detection of changes from a reference sequence.

The method may be performed in respect of a single subject polymer unit or plural subject polymer units in the sequence of polymer units. For example the method may be applied a subject polymer unit forming part of a predetermined motif, for example a CpG site which is known to have a relatively high likelihood of modification.

According to a second aspect of the present invention, there is provided a computer program comprising instructions which, when the program is executed by a computer, cause the computer to carry out a method in accordance with the first aspect of the present invention. The computer program may be stored on a computer storage medium.

According to a third aspect of the present invention, there is provided a method of analysing a polymer comprising: deriving a measurement signal from the polymer during translocation of the polymer with respect to a nanopore, the polymer comprising a sequence of polymer units; and analysing the measurement signal using a method in accordance with the first aspect of the present invention.

According to a fourth aspect of the present invention, there is provided an analysis apparatus comprising a processor configured to carry out a method in accordance with the first aspect of the present invention. The analysis apparatus may form part of a nanopore measurement and analysis system that further comprises a measurement system arranged to derive a measurement signal from a polymer during translocation of the polymer with respect to a nanopore.

According to a fifth aspect of the present invention, there is provided a method of training a slice machine learning system to provide an output representing an estimate of the identity of a subject polymer unit of interest in a polymer by supplying the machine learning system with training signals comprising plural pairs of a training sequence slice around a subject polymer unit in a sequence of polymer units of a polymer, and a training signal slice of a measurement signal measured from the polymer during translocation of the polymer with respect to a nanopore.

To allow better understanding, embodiments of the present invention will now be described by way of non-limitative example with reference to the accompanying drawings, in which:

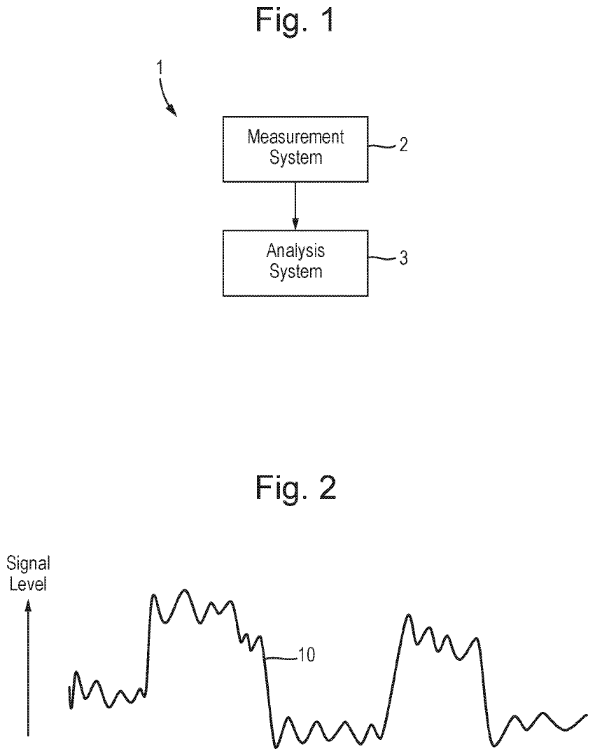

FIG. 1 is a schematic diagram of a nanopore measurement and analysis system;

FIG. 2 is a plot of a typical measurement signal over time;

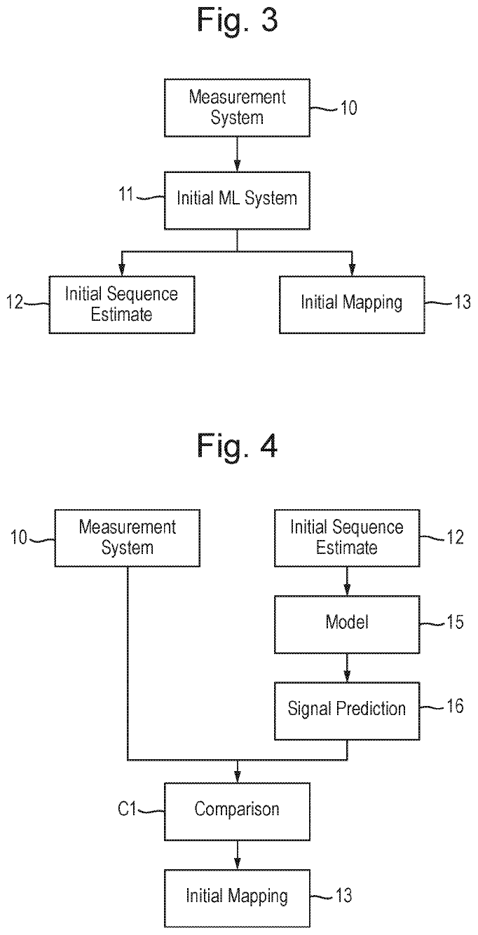

FIG. 3 is a flowchart of a method of deriving an initial sequence estimate using an initial machine learning system;

FIG. 4 is a flowchart illustrating a method of deriving an initial mapping between an initial sequence estimate and a measurement signal;

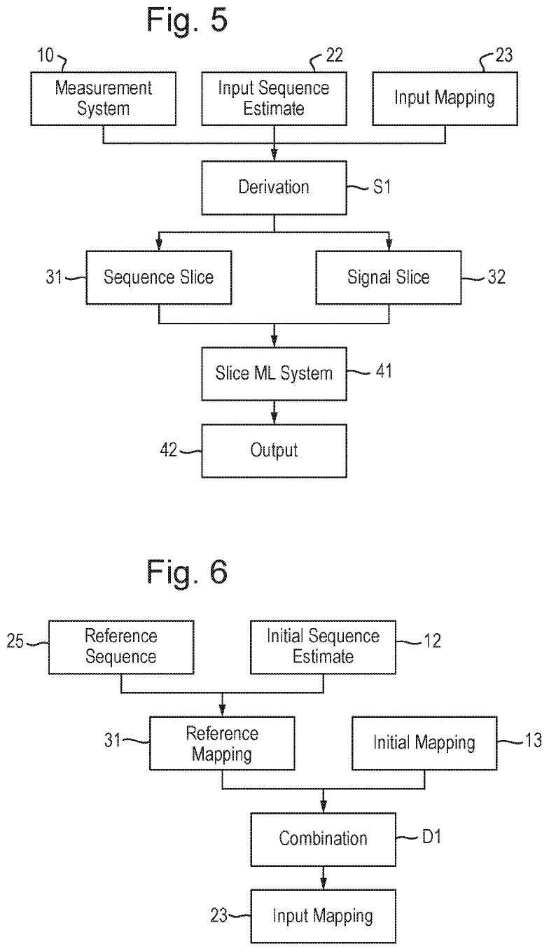

FIG. 5 is a flowchart of a method of deriving an output using a slice machine learning system;

FIG. 6 is a flowchart illustrating a method of deriving an input mapping in an example where the input sequence estimate is a reference sequence;

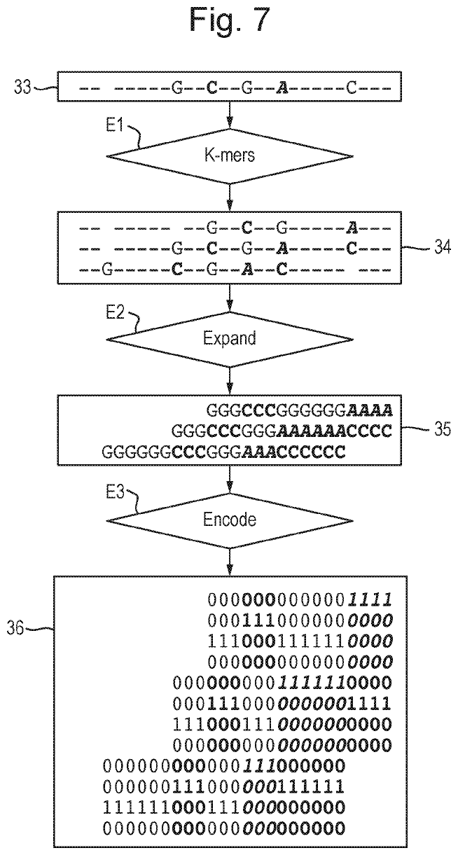

FIG. 7 is a diagram illustrating a method of generating a sequence slice that is mapped to a signal slice;

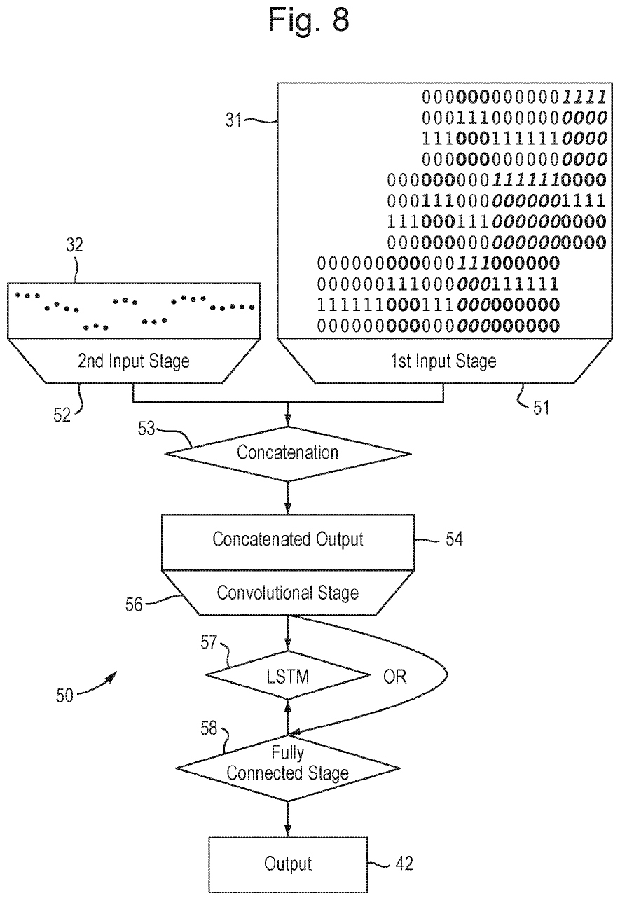

FIG. 8 is a diagram illustrating an example of the slice machine learning system that is a neural network; and

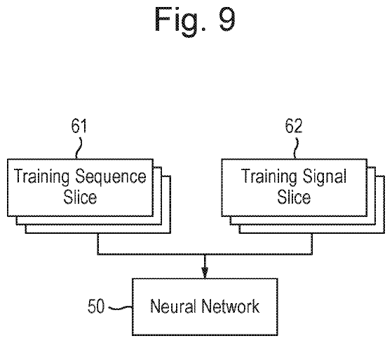

FIG. 9 is a diagram illustrating training of a neural network as an example of the slice machine learning system.

FIG. 1 illustrates a nanopore measurement and analysis system 1 comprising a measurement system 2 and an analysis system 3. The measurement system 2 derives a measurement signal 10 from a polymer comprising a series of polymer units during translocation of the polymer with respect to a nanopore. The analysis system 3 performs a method of analysing the measurement signal 10 to derive an estimate of the series of polymer units.

In general, the polymer may be of any type, for example a polynucleotide (or nucleic acid), a polypeptide such as a protein, or a polysaccharide. The polymer may be natural or synthetic. The polynucleotide may comprise a homopolymer region. The homopolymer region may comprise between 5 and 15 nucleotides.

In the case of a polynucleotide or nucleic acid, the polymer units may be nucleotides. The nucleic acid is typically deoxyribonucleic acid (DNA), ribonucleic acid (RNA), or a synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety. The nucleic acid may be single-stranded, be double-stranded or comprise both single-stranded and double-stranded regions. The nucleic acid may comprise one strand of RNA hybridised to one strand of DNA. Typically cDNA, RNA, GNA, TNA or LNA are single stranded.

The polymer units may be any type of nucleotide. The nucleotide can be naturally occurring or artificial. For instance, the method may be used to verify the sequence of a manufactured oligonucleotide. A nucleotide typically contains a nucleobase, a sugar and at least one phosphate group. The nucleobase and sugar form a nucleoside. The nucleobase is specifically adenine, guanine, thymine, uracil and cytosine. The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate.

The polymer units may be canonical polymer units. For example in the case that the polymer is a DNA polynucleotide, the canonical bases are adenine (A), cytosine (C), guanine (G), and thymine (T). By contrast ribonucleic acid (RNA) comprises the canonical bases A. C and G, with uracil (U) in place of thymine.

The nucleotide can be a modified polymer unit, such as a damaged or epigenetic base. For instance, the nucleotide may comprise a pyrimidine dimer. Such dimers are typically associated with damage by ultraviolet light and are the primary cause of skin melanomas. The nucleotide can be labelled or modified to act as a marker with a distinct signal. This technique can be used to identify the absence of a base, for example, an abasic unit or spacer in the polynucleotide. The method could also be applied to any type of polymer.

In the case of a polypeptide, the polymer units may be amino acids that are naturally occurring or synthetic.

In the case of a polysaccharide, the polymer units may be monosaccharides.

Particularly where the measurement system 2 comprises a nanopore and the polymer comprises a polynucleotide, the polynucleotide under investigation may range in length from typically 500 nucleotides (500 b) to greater than 2 Mb in length. However polynucleotides of shorter length may be measured with the lower limit estimated to be around 10-20 bases depending upon the length of the nanopore channel, which would include mRNA, tRNA and cfDNA.

The nature of the measurement system 2 and the resultant measurement signal 10 is as follows.

The measurement system 2 is a nanopore system that comprises one or more nanopores. In a simple type, the measurement system 2 has only a single nanopore, but a more practical measurement systems 2 employ many nanopores, typically in an array, to provide parallelised collection of information.

The measurement signal 10 may be recorded during translocation of the polymer with respect to the nanopore, typically through the nanopore.

The nanopore is a pore, typically having a size of the order of nanometres, that may allow the passage of polymers therethrough.

The nanopore may be a protein pore or a solid state pore. The dimensions of the pore may be such that only one polymer may translocate the pore at a time.

Where the nanopore is a protein pore, it may have the following properties.

The biological pore may be a transmembrane protein pore. Transmembrane protein pores for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, lysenin, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin. The transmembrane pore may be derived from Msp or from α-hemolysin (α-HL). The transmembrane pore may be derived from lysenin. Suitable pores derived from lysenin are disclosed in WO 2013/153359. Suitable pores derived from MspA are disclosed in WO-2012/107778. The pore may be derived from CsgG, such as disclosed in WO-2016/034591 and WO2019/002893, both herein incorporated by reference in their entirety. The pore may be a DNA origami pore.

The protein pore may be a naturally occurring pore or may be a mutant pore.

The protein pore may be inserted into an amphiphilic layer such as a biological membrane, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed in Gonzalez-Perez et al., Langmuir, 2009, 25, 10447-10450, WO2014/064444, or U.S. Pat. No. 6,723,814 herein incorporated by reference in its entirety. Alternatively, a protein pore may be inserted into an aperture provided in a solid state layer, for example as disclosed in WO2012/005857.

A suitable apparatus for providing an array of nanopores is disclosed in WO-2014/064443. The nanopores may be provided across respective wells wherein electrodes are provided in each respective well in electrical connection with an ASIC for measuring current flow through each nanopore. A suitable current measuring apparatus may comprise the current sensing circuit as disclosed in WO-2016/181118.

The nanopore may comprise an aperture formed in a solid state layer, which may be referred to as a solid state pore. The aperture may be a well, gap, channel, trench or slit provided in the solid state layer along or into which analyte may pass. Such a solid-state layer is not of biological origin. In other words, a solid state layer is not derived from or isolated from a biological environment such as an organism or cell, or a synthetically manufactured version of a biologically available structure. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO-2009/035647, WO-2011/046706 or WO-2012/138357. Suitable methods to prepare an array of solid state pores is disclosed in WO-2016/187519.

Such a solid state pore is typically an aperture in a solid state layer. The aperture may be modified, chemically, or otherwise, to enhance its properties as a nanopore. A solid state pore may be used in combination with additional components which provide an alternative or additional measurement of the polymer such as tunnelling electrodes (Ivanov A P et al., Nano Lett. 2011 Jan. 12; 11 (1):279-85), or a field effect transistor (FET) device (as disclosed for example in WO-2005/124888). Solid state pores may be formed by known processes including for example those described in WO-00/79257.

The nanopore may be a hybrid of a solid state pore with a protein pore.

The measurement system 2 takes a series of measurements of a property that depends on the polymer units translocating with respect to the pore may be measured. The series of measurements form the measurement signal 10.

The property that is measured may be associated with an interaction between the polymer and the pore. Such an interaction may occur at a constricted region of the pore.

In one type of measurement system 2, property that is measured may be the ion current flowing through a nanopore. These and other electrical properties may be measured using standard single channel recording equipment as describe in Stoddart D et al., Proc Natl Acad Sci, 12; 106 (19):7702-7, Lieberman K R et al, J Am Chem Soc. 2010; 132 (50):17961-72, and WO-2000/28312. Alternatively, measurements of electrical properties may be made using a multi-channel system, for example as described in WO-2009/077734. WO-2011/067559 or WO-2014/064443.

Ionic solutions may be provided on either side of the membrane or solid state layer, which ionic solutions may be present in respective compartments. A sample containing the polymer analyte of interest may be added to one side of the membrane and allowed to move with respect to the nanopore, for example under a potential difference or chemical gradient. The measurement signal 10 may be derived during the movement of the polymer with respect to the pore, for example taken during translocation of the polymer through the nanopore. The polymer may partially translocate the nanopore.

In order to allow measurements to be taken as the polymer translocates through a nanopore, the rate of translocation can be controlled by a polymer binding moiety. Typically the moiety can move the polymer through the nanopore with or against an applied field. The moiety can be a molecular motor using for example, in the case where the moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, helicases, exonucleases, single stranded and double stranded binding proteins, and topoisomerases, such as gyrases. For other polymer types, moieties that interact with that polymer type can be used. The polymer interacting moiety may be any disclosed in WO-2010/086603, WO-2012/107778, and Lieberman K R et al, J Am Chem Soc. 2010; 132 (50):17961-72), and for voltage gated schemes (Luan B et al., Phys Rev Lett. 2010; 104 (23):238103). The rate of translocation of the polymer through the nanopore may be controlled by a voltage control pulse to step the polymer through the nanopore such as disclosed in WO2019/006214. Translocation of the polymer may be controlled by a molecular hopper such as disclosed by WO2020/016573.

The polymer binding moiety can be used in a number of ways to control the polymer motion. The moiety can move the polymer through the nanopore with or against the applied field. The polynucleotide binding enzyme does not need to display enzymatic activity as long as it is capable of binding the target polynucleotide and controlling its movement through the pore. For instance, the enzyme may be modified to remove its enzymatic activity or may be used under conditions which prevent it from acting as an enzyme. Such conditions are discussed in more detail below.

The polynucleotide binding enzyme may be a Dda helicase such as disclosed in WO2015055981, hereby incorporated by reference in its entirety.

Translocation of the polymer through the nanopore may occur, either cis to trans or trans to cis, either with or against an applied potential. The translocation may occur under an applied potential which may control the translocation. The binding enzyme is typically held against the cis or trans opening of the nanopore during translocation of the polynucleotide through the nanopore under an applied potential.

Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as a molecular brake slowing down the movement of a polynucleotide through the pore. Any moieties, techniques or enzymes described in WO-2012/107778 or WO-2012/033524 could be used to control polymer motion.

However, the measurement system 2 may be of alternative types that comprise one or more nanopores.

Similarly, the properties that are measured may be of types other than ion current. Some examples of alternative types of property include without limitation: electrical properties and optical properties. A suitable optical method involving the measurement of fluorescence is disclosed by J. Am. Chem. Soc. 2009, 131 1652-1653. Possible electrical properties include: ionic current, impedance, a tunnelling property, for example tunnelling current (for example as disclosed in Ivanov A P et al., Nano Lett. 2011 Jan. 12; 11 (1):279-85), and a FET (field effect transistor) voltage (for example as disclosed in WO2005/124888). One or more optical properties may be used, optionally combined with electrical properties (Soni G V et al., Rev Sci Instrum. 2010 January; 81 (1): 014301). The property may be a transmembrane current, such as ion current flow through a nanopore. The ion current may typically be the DC ion current, although in principle an alternative is to use the AC current flow (i.e. the magnitude of the AC current flowing under application of an AC voltage).

In some types of the measurement system 2, the measurement signal 10 may be characterised as comprising measurements from a series of events, where each event provides a group of measurements. FIG. 2 illustrates a typical example of such a measurement signal 10 in the case of measurement of current. The group of measurements from each event have a level that is similar, although subject to some variance. This may be thought of as a noisy step wave with each step corresponding to an event. The events may have biochemical significance, for example arising from a given state or interaction of the measurement system 2. This may in some instances arise from translocation of the polymer through the nanopore occurring in a ratcheted manner. However, this type of signal is not produced by all types of measurement system and the methods described herein are not dependent on the type of signal. For example, when translocation rates approach the measurement sampling rate, for example, measurements are taken at 1 times, 2 times, 5 times or 10 times the translocation rate of a polymer unit, events may be less evident or not present, compared to slower sequencing speeds or faster sampling rates.

In addition, where events are present, typically there is no a priori knowledge of number of measurements in the group, which varies unpredictably. These factors of variance and lack of knowledge of the number of measurements can make it hard to distinguish some of the groups, for example where the group is short and/or the levels of the measurements of two successive groups are close to one another.

The group of measurements corresponding to each event typically has a level that is consistent over the time scale of the event, but for most types of the measurement system 2 will be subject to variance over a short time scale. Such variance can result from measurement noise, for example arising from the electrical circuits and signal processing, notably from the amplifier in the particular case of electrophysiology. Such measurement noise is inevitable due the small magnitude of the properties being measured. Such variance can also result from inherent variation or spread in the underlying physical or biological system of the measurement system 2, for example a change in interaction, which might be caused by a conformational change of the polymer.

Most types of the measurement system 2 will experience such inherent variation to greater or lesser extents. For any given types of the measurement system 2, both sources of variation may contribute or one of these noise sources may be dominant.

With increase in the sequencing rate, being the rate at which polymer units translocate with respect to the nanopore, then the events may become less pronounced and hence harder to identify, or may disappear. Thus, analysis methods that rely on detecting such events detection may become less efficient at as the sequencing rate increases.

However, the methods disclosed herein are not dependent on detecting such events. The methods described below are effective even at relatively high sequencing rates, including sequencing rates at which the polymer translocates at a rate of at least 10 polymer units per second, preferably 100 polymer units per second, more preferably 500 polymer units per second, or more preferably 1000 polymer units per second.

The sample rate is the rate of measurements in the signal. Typically, the sample rate is higher than the sequencing rate. For example, the sample rate may be in a range from a 100 Hz to 30 kHz, but this is not limitative. In practice the sample rate may depend on the nature of the measurement system 2.

The analysis system 3 may be physically associated with the measurement system 2, and may also provide control signals to the measurement system 2. In that case, the nanopore measurement and analysis system 1 comprising the measurement system 2 and the analysis system 3 may be arranged as disclosed in any of WO-2008/102210, WO-2009/07734, WO-2010/122293, WO-2011/067559 or WO2014/04443.

Alternatively, the analysis system 3 may implemented in a separate apparatus, in which case the series of measurement is transferred from the measurement system 2 to the analysis system 3 by any suitable means, typically a data network. For example, one convenient cloud-based implementation is for the analysis system 3 to be a server to which input signals are supplied over the internet.

The analysis system 3 may be implemented by a computer apparatus executing a computer program or may be implemented by a dedicated hardware device, or any combination thereof. In either case, the data used by the method is stored in a memory in the analysis system 3.

In the case of a computer apparatus executing a computer program, the computer apparatus may be any type of computer system but is typically of conventional construction. The computer program may be written in any suitable programming language. The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory.

In the case of the computer apparatus being implemented by a dedicated hardware device, then any suitable type of device may be used, for example an FPGA (field programmable gate array) or an ASIC (application specific integrated circuit). In a preferred embodiment, portions of the computer program may be implemented using hardware amenable to parallelisation of calculations such as a Graphics processing unit (GPU).

A method of using the nanopore measurement and analysis system 1 is performed as follows.

The measurement signal 10 is derived using the measurement system 2. For example, the polymer is caused to translocate with respect to the pore, for example through the pore, and the measurement signal 10 is derived during the translocation of the polymer. The polymer may be caused to translocate with respect to the pore by providing conditions that permit the translocation of the polymer, whereupon the translocation may occur spontaneously. The analysis system 3 performs a method of analysing the measurement signal 10 as will now be described.

The measurement signal 10 is a raw nanopore signal that represents measurements taken by the measurement signal. Typically, the measurement system 2 will take measurements using a sensor and derive values that are output from a data acquisition device (DAQ), for example having a digital to analog converter (DAC), digital integer values representing the signals read from the nanopore sequencing device. Typically, the absolute level of the output from the DAQ will depend on the electronics used. Accordingly to make the signal more useful, and in common with the vast majority of known nanopore analysis systems, the measurement signal 10 is normalised prior to the subsequent processing described below

Several methods to perform this signal normalization process are known in the art. For example, such normalisation may involve centering the measurement signal 10 on zero and scaling the measurement signal 10 to an approximate standard deviation of one. Alternatively, the normalization may aiming to reflect the physical electric current measurements (in units of amperes or picoamperes). Other signal normalization processes are also known. Optionally, the signal normalisation process may change the sampling rate.

In this context, the term “raw” when used to describe the measurement signal 10 refer to the normalisation signal 10 after such normalisation, and not to the output from the DAQ.

FIG. 3 illustrates a method of using an initial machine learning system 11 to derive an initial sequence estimate 12 of the sequence of polymer units of the polymer from which the measurement signal 10 is taken. Specifically, the measurement signal 10 is supplied as an input to the initial machine learning system 11, which is trained to provide an output that is the initial sequence estimate 12. In general, the initial machine learning system 11 may take any suitable form, but is typically a neural network. For example, the initial machine learning system 11 may be a neural network of the type disclosed in: Hochreiter, S. and Schmidhuber, J., 1997. Long short-term memory. Neural computation, 9 (8), pp. 1735-1780; Cho, K., Van Merrienboer, B., Bahdanau, D. and Bengio, Y., 2014. On the properties of neural machine translation: Encoder-decoder approaches. arXiv preprint arXiv: 1409.1259; Kriman, S., Beliaev, S., Ginsburg, B., Huang. J., Kuchaiev, O., Lavrukhin, V., Leary, R., Li, J. and Zhang, Y., 2020 May. Quartznet: Deep automatic speech recognition with 1d time-channel separable convolutions. In ICASSP 2020-2020 IEEE International Conference on Acoustics. Speech and Signal Processing (ICASSP) (pp. 6124-6128). IEEE; or Teng. H., Cao, M. D., Hall, M. B., Duarte, T., Wang, S. and Coin, L. J., 2018. Chiron: translating nanopore raw signal directly into nucleotide sequence using deep learning. GigaScience. 7 (5), and to which standard training techniques are applied.

The initial sequence estimate 12 may be a categorical output. It may represent an estimate of the identity of polymer units in the sequence between categories comprising a set of predetermined canonical polymer units. For example in the case that the polymer unit is a DNA polynucleotide, the canonical nucleotides may be the four bases adenine (A), cytosine (C), guanine (G), and thymine (T). In general, such a categorical output may be implemented as a vector of probabilities over the categories. However, for use in the subsequent method, a hard call is made. That is the most likely category. e.g. the most likely canonical polymer unit is selected and represented in the initial sequence estimate 12.

Optionally, the initial machine learning system 11 may also output an initial mapping 13 between the measurement signal 10 and the initial sequence estimate 12. Typically, such a initial mapping 13 is inherently generated during the operation of a machine learning system such as a neural network. It is often referred to as the “move table” in nanopore basecalling documentation and prior art. Generally, this initial mapping 13 is discarded as the generally desired output is simply the sequence estimate. However, generally the initial mapping 13 can be obtained and output from the initial machine learning system 11, when needed.

The initial mapping 13 simply describes the originating position of each polymer unit of the initial sequence estimate 12 with corresponding samples of the measurement signal 10. The initial mapping 13 may be encoded in several equivalent forms. For example, an array of indices the length of the initial sequence estimate 12 and with elements corresponding to the position of samples of the measurement signal 10 would completely represent this mapping. Equivalently the length, in number of signal positions, of each polymer unit of the initial sequence estimate 12 would completely describe this mapping in a more compact manner.

It is assumed that the position of a polymer unit within the measurement signal 10 is not before the position of the polymer unit. In other words, a polymer unit later in the initial sequence estimate 12 may not be assigned a position earlier in the measurement signal 10. It is also assumed that each input sequence polymer unit is assigned a starting position within the signal array, implying that many signal positions may be assigned to a single sequence base, and this is often the case.

As an alternative to the initial mapping 13 being output from the initial machine learning system 11, the initial mapping 13 may be derived from the measurement signal 10 and the initial signal estimate 12 themselves. Several methods are described in the prior art for the generation of such a sequence-to-signal mapping, for example in: Stoiber, M. H. et al. De novo Identification of DNA Modifications Enabled by Genome-Guided Nanopore Signal Processing. bioRxiv (2016); or Simpson, Jared T., et al. “Detecting DNA cytosine methylation using nanopore sequencing.” nature methods 14.4 (2017): 407-410. Such methods may be applied here.

By way of example, FIG. 4 illustrates a suitable method of deriving the initial mapping 13 from the measurement signal 10 and the initial sequence estimate 12 that may be applied, as follows.

The initial sequence estimate 12 is supplied to a model 15 which is a model of the measurement system 2 that was used to provide the measurement signal 10. The model generates a signal prediction 16, being a prediction of the signal predicted by the model 15 to be generated from the initial sequence estimate 12. The model 15 may use a small window of polymer units (a “k-mer”) to determine the expected signal level at a particular sequence position.

In comparison step C1, the signal prediction 16 is compared with the measurement signal 10 to derive the initial mapping 13 based on that comparison. Since the expected signal levels are directly attributed to the polymer units of the initial sequence estimate 12, this defines the initial mapping 13. Generally, a dynamic programming algorithm may be used here.

Further processing the measurement signal 10 performed after the use of the initial machine learning system 11 will now be described.

FIG. 5 illustrates a method using a slice machine learning system 41, as follows.

There are three inputs to this method, namely 1) the measurement signal 10, 2) an input sequence estimate 22, and 3) an input mapping 23 between the measurement signal 10 and the input sequence estimate 22. The form of the input sequence estimate 22 is further discussed below, but in general terms is based on the initial sequence estimate 12 output from the initial machine learning system 11.

In a derivation step S1, there are derived two slices, namely 1) a sequence slice 31 and a signal slice 32, which are input into the slice machine learning system 41. The sequence slice 31 is derived from a slice of the input sequence estimate 22 around a subject polymer unit in the sequence of polymer units. The signal slice 32 is a slice of the measurement signal 10. Importantly, the sequence slice 31 and the signal slice 32 are mapped to each other by the input mapping 23 between the measurement signal 10 and the input sequence estimate 22.

To summarise this at a high level, this method involves the input of a sequence slice 31, which is a canonical sequence, and a measurement slice 32 of the measurement signal 10, which is a raw measurement signal, directly into the slice machine learning system 41. This may be referred to as a multi-headed input. In contrast, known canonical basecalling systems are typically based on a single-headed neural network as only a single form of data is input into the neural network, namely the raw nanopore signal. To enable multi-headed input, the sequence slice 31 and the signal slice 32 are presented in a manner described further below.

Reverting to the input sequence estimate 22, this may take different forms which are derived as follows.

In one form, the input sequence estimate 22 may simply be the initial sequence estimate 12 provided as the output of the initial machine learning system 11 that was supplied with the measurement signal slice 10 as an input. This is the simplest form of the input sequence estimate 22 and results in the slice machine learning system 41 improving the accuracy and/or information content as compared to mere consideration of the initial sequence estimate 12. In this case, the input mapping 23 between the measurement signal 10 and the input sequence estimate 22 is simply the initial mapping 13 between the measurement signal 10 and the initial sequence estimate 12. Herein, this alternative is referred to as “basecall anchoring” in that in some embodiments it refers to nucleobases. (although the term “basecall” here does not imply the polymer units are bases in all cases and the term could equally apply to other types for polymer unit, for example protein monomers).

In another form, the input sequence estimate 22 may be a reference sequence in respect of the polymer. Herein, this alternative is referred to as “reference anchoring”. The reference sequence for the polymer may be obtained from a standard resource or library, for example a resource provided by the National Center for Biotechnology Information (NCBI) or an Ensembl resource. Alternatively, the reference sequence may be produced from an aggregation (or consensus) of the measurement signals 10 from the same sample, or from a known ground truth in cases of synthetic polymers.

The initial sequence estimate 12 generally contains some errors. It has been shown that, particularly when using an initial machine learning system 11 of relatively low quality (for example to use less compute resources or computation time), the accuracy of the estimation by the slice machine learning system can be greatly improved by transferring from basecall anchoring to reference anchoring.

In this case, the input mapping 23 between the measurement signal 10 and the input sequence estimate 22, i.e. the reference sequence, may be obtained by a process known as genomic or reference alignment.

An example of such a method is shown in FIG. 6 and performed as follows using: 1) the reference sequence 25; 2) the initial sequence estimate 12, which may be derived as described above, and 3) the initial mapping 13 between the measurement signal 10 and the initial sequence estimate 12, which may be derived by any of the techniques described above.

There is derived a reference mapping 26 between the reference sequence 25 and the initial sequence estimate 12. This is achieved by assigning the estimated polymer units of the initial sequence estimate 12 to respective polymer units of the reference sequence 25. Within the bounds of the matched portions of these two sequences, an alignment is determined. The reference mapping at the level of polymer units maps stretches of matched positions between the estimated polymer units of the initial sequence estimate 12 and reference positions within the reference sequence 25, as well as the locations of any skipped polymer units within the reference sequence 25 and the initial sequence estimate 12.

In combination step D1 the reference mapping 26 is combined with the initial mapping 13 to derive the input mapping 23. This step reconstructs the sequence to signal mapping assigned to the reference sequence 25 that is used as the input sequence estimate 22. For positions within the reference sequence with a direct mapping to a position in the estimated polymer units of the initial sequence estimate 12, the signal position is transferred to the corresponding position in the reference sequence 25. For positions within the reference sequence 25 between stretches of matching positions any valid indices within the measurement signal 10 are allowed. Specifically, the signal position assignments within unmatched reference regions should be greater than or equal to the last position before the unmatched reference region and should be less than or equal to the first matched reference position after the unmatched reference region. This procedure should be carried out at each stretch of unmatched reference sequence 25 to produce a full mapping 22 that can be applied to the slice machine learning system 41, in the same manner as for basecall anchoring.

For reference-anchoring, the goal is to make predictions against subject polymer units from the reference sequence. The reference sequence is provided the full extent of the region determined as matching based on the reference alignment. In some cases, this may be composed of discontinuous sections of the reference.

We now revert to the method of using the slice machine learning system 41 shown in FIG. 5.

As mentioned above, the sequence slice 31 and the signal slice 32 are derived in derivation step S1 as slices around a subject polymer unit being considered.

The method may be applied to a single subject polymer unit in the input sequence estimate 22 or repeatedly to plural subject polymers being all or any subset of the polymer units in the input sequence estimate 22

For example, the method may performed for a subject polymer unit forming part of a predetermined motif comprising plural canonical polymer units. Often a motif (a short pattern of polymer units (e.g. nucleotides) which may include positions of ambiguity allowing several polymer units or variable widths of polymer units used to identify the relevant subject polymer units. For example, the “CG” motif, also referred to as a CpG site, is the most common motif in which methylation occurs in most mammals, and may form a motif used herein.

Examples of the derivation of the sequence slice 31 and the signal slice 32 in derivation step S1 will now be described in more detail. As mentioned above, the sequence slice 31 is derived from a slice of the input sequence estimate 22 around a subject polymer unit and the signal slice 32 is a slice of the measurement signal 10, the sequence slice 31 and the signal slice 32 being mapped to each other by the input mapping 23. There are various ways to achieve this, for example as follows.

The measurement signal 10, the input sequence estimate 22, and the input mapping 23 are generally provided as a full sequencing read corresponding to an entire nanopore read, which is typically very long, for example consisting of tens to millions of individual polymer units for some types of measurement system 2. However, derivation step S1 provides the sequence slice 31 and the signal slice 32 with corresponding lengths that are selected to suitable accuracy to for the slice machine learning system 41.

In one approach, the signal slice 32 is a predetermined length of the measurement signal 10 around a position in the measurement signal 10 that is mapped to the subject polymer unit. In this case, once subject polymer unit within the input sequence estimate 22 is identified, the location within the measurement signal 10 to which the subject polymer unit is assigned from the input mapping 23. The center of this stretch of the measurement signal 10 is defined as the center of the region of interest. From this position a fixed width of signal is extracted using a user defined range before and after this position.

In this case, the predetermined length of the measurement signal 10 may, for example, be in a range from 20 sample points to 1000 sample points, for example 100 sample points. Larger lengths of the measurement signal 10 may be more than 1000 sample points. The signal slice 32 may be arranged symmetrically or asymmetrically around the sample point that is mapped to the subject polymer unit.

In addition to extracting the signal slice 32 from this region, the sequence slice 31 is selected as the polymer units mapped to the stretch of the signal slice 32 by the input mapping 23. Accordingly, the length of the sequence slice 31 varies for different subject polymer units.

In another approach, the sequence slice 31 is a predetermined length of the input sequence estimate 22, that is a predetermined number of polymer units. In this case, once the sequence slice 31 has been extracted, the signal slice 32 is derived as the portion of the measurement signal 10 that is mapped to the sequence slice 31 by the input mapping 23. Accordingly, the length of the signal slice 32 varies for different subject polymer units.

In this case, the predetermined number of polymer units may be in a range from 1 polymer unit to 100 polymer units. The range of polymer units to be considered may be dependent on the type of nanopore used.

Optionally, the sequence slice 31 may be selected to consider nanopore kinetics, as follows. When the rate of translocation of a polynucleotide through a nanopore is controlled by a molecular brake in the form of an enzyme, it is believed for example that modified bases affect the enzyme kinetics such as the kinetics of unwinding of double stranded polynucleotides by certain helicases. In the case of a helicase as the binding enzyme which may serve to unwind double stranded DNA and control passage of a resultant single stranded DNA strand through the nanopore, consideration of those nucleotides within the enzyme binding region may further provide information about the signal.

As such, it may be of value to provide such information to nanopore modified base detection algorithms. This may be achieved by the sequence slice 31 being derived in a manner that one or more nucleotides of the sequence slice 31 are within a region of the enzyme acting as a molecular brake to controls translocation of the polymer.

This may improve accuracy compared to providing the same size of signal, but without including the signal when the base of interest is in the molecular brake. Note that this may provide improved performance over alternative nanopore modified base detection algorithms which attempt to provide this information via summaries of the raw nanopore signal as signal to sequence assignment/alignment algorithms are often quite error prone. As noted in other sections passing the raw nanopore signal into the neural network may allow for improved performance bypassing issues with sequence to signal alignments.

It has been shown that changes in the signal may be influenced most due to interaction of the nucleotides with one or more constrictions of the nanopore, a constriction being a region of the internal lumen of the nanopore of narrow cross-section, see for example, FIG. 1 of Butler et al, Proceedings of the National Academy of Sciences 105 (52), 20647-20652 which shows an MspA nanopore with an inner narrow constriction at the D90N/D91N region and FIGS. 1 and 2 of WO2016/034591 which shows the inner constriction region of CsgG nanopore However interaction with other regions of the nanopore can affect the signal and nucleotides external to the nanopore are also believed to have an influence on the measured signal. In use, the binding enzyme is typically held against the cis or trans opening of the nanopore during translocation of the polynucleotide through the nanopore under an applied potential. Thus nucleotides immediately outside of the lumen of the nanopore are typically within the region of the binding enzyme, for example with dDA helicase as the polynucleotide binding enzyme and CsgG as the nanopore, the distance between the enzyme and the constriction is estimated at between 10 and 14 bases (or approximately 100 to 140 signal points). Signal point measurements depend on several factors and may vary drastically from these values for other pore chemistries).

FIG. 7 illustrates a particular method of generating the sequence slice 31 in an appropriate form for input to the slice machine learning system 41 mapped to the signal slice 32. This procedure is intended to maximize the information presented to the slice machine learning system 41.

Initially, a first signal slice 33 is extracted as a slice of the input sequence estimate 22, which, for non-limitative, illustrative purposes, has in FIG. 7 a particular sequence of nucleotides that are different canonical nucleotides selected from the four bases A, C, G or T. Graphically in FIG. 7 the input mapping 23 is represented by dashes. In particular, each element of the first sequence slice 33 that is either a nucleotide or a dash corresponds to a respective sample point in the corresponding signal slice 32, in accordance with the input mapping 23.

In step E1, the first sequence slice 33 is encoded into a second sequence slice 34 by replacing each polymer unit by a respective k-mer, so that the second sequence slice 34 is a sequence of k-mers corresponding to respective polymer units in the first input slice 33. Thus, compared to the first sequence slice 33, the second sequence slice 34 has the same length but increased dimensionality so that each element of the second sequence slice 34 is a vector of k dimensions (k being 3 in FIG. 7, by way of non-limitative example). Each k-mer in the second sequence slice 34 comprises a group of k polymer units (arranged vertically in FIG. 7), where k is a plural integer. Each k-mer includes a) the respective polymer unit (along the middle dimension in FIG. 7), and b) (k−1) polymer units that are adjacent to the respective polymer unit in the input sequence estimate 23. The (k−1) adjacent polymer units symmetrical around the respective polymer unit in FIG. 7, but as an alternative (k−1) adjacent polymer units be selected asymmetrically. It should be noted that this encoding requires a fixed number of polymer units before and after the first signal slice 33 to enable the construction of the k-mers.

This change from polymer units to k-mers effectively provides additional contextual information to the individual polymer. These k-mers may be thought of as representing the portion of the polymer which physically interacted with the nanopore at a particular position within the signal, although that is conceptual and may not be a complete description of any particular measurement system 2. Nonetheless, in the case that the polymer translocates through the nanopore, k may have has a value selected so that the length of the k-mer is greater than the length of the nanopore lumen through which the polymer translocates.

The use of k-mers in this way has been shown to improve the accuracy of the estimation performed by the slice machine learning system 41. In general, the k may have any value that provides such an improvement, noting that increasing k increases the size of the data without significantly increasing the computational cost. In some examples, k may have a value in a range from 3 to 50, but higher values are also possible.

As an alternative, step E1 may be omitted so that the following steps are performed on the first sequence slice 33, although that is likely to reduce the accuracy of the estimation performed by the slice machine learning system 41.

In step E2, the second sequence slice 34 is expanded into a third sequence slice 35, so that it has the same length as the signal slice 32. In this example, the expansion is performed by repetition padding which is shown graphically in FIG. 7 as a replacement of the dashes by the k-mer that preceded them. This expansion allows efficient design of the slice machine learning system 41, described below.

In step E3, the third sequence slice 35 is binary encoded into a final sequence slice 36, which is used as the input sequence slice 31 to the slice machine learning system 41. The binary encoding encodes each polymer unit in binary format, in this example using a one-hot encoding (“1000” for A; “0100” for C; “0010” for G; “0001” for T; and “0000” for unknown or missing bases). For each position in the third sequence slice 35, the k vectors of length 4 for the k polymer units of the k-mer are concatenated to form a vector of length 4k.

The slice machine learning system 41 is supplied with the sequence slice 31 and the signal slice 32 of equal length as a double-headed input. The slice machine learning system 41 has been trained to provide an output 42 representing an estimate of the identity of the subject polymer unit. The output 42 is a categorical output. That is, the output 42 estimates the identity of the subject polymer unit as between a set of categories. Such a categorical output may be implemented as a vector of probabilities over the categories. The slice machine learning system 41 is trained to maximise the probability for the correct output category and minimise the probability for the incorrect output categories. To optimize categorical output type, the cross-entropy loss is generally used in the slice machine learning system 41 that is described further below, although there are other loss functions that could be applied to such a categorical output 42.

The nature of the categories represented by the output 42 may take various forms, depending on the application.

In some types of embodiment concerned with detection of modified forms of a canonical polymer unit, the categories represented by the output 42 may be a canonical polymer unit and at least one modified form of the canonical polymer unit. By way of non-limitative example, where the polymer is DNA and the polymer units are nucleotides, then the canonical polymer unit may be cytosine or adenosine, and the at least one modified form of the canonical polymer unit is at least one of 5-methyl-cytosine and 5-hydroxymethyl-cytosine in the case that the canonical polymer unit is cytosine or is 6-methyl-adenosine in the case that the canonical polymer unit is adenosine.

To consider this more generally, the modified bases 5-methylcytosine (5mC) and 5-hydroxymethyl-cytosine are well-known epigenetic mark that regulates transcription of the genome (the switching on and off of the mechanism by which DNA is copied into messenger RNA (mRNA), which is involved in protein synthesis. Accordingly, methylation is a type of modification that the categorical output 42 may represent and is important because it is generally the most biologically relevant.

However, the categorical output 42 may in general represent any type of modification without restriction to methylation. By way of example, another modification that the categorical output 42 may represent is oxidation, for example oxidation of methylated cytosine (5-mC) to 5-hydroxymethylcytosine (5-hmC), 5-formylcytosine (5-fC), 5-carboxylcytosine (5-caC), and methylation of adenine (A) to N6-methyladenine (6-mA), which are being identified as important epigenetic regulators.

In the case that the polymer is RNA, modifications are more prevalent and recent work has shown that it plays a role in regulating mRNA stability. The stability of mRNA effects control of gene expression and can effect various cellular and biological processes. To date, hundreds of RNA modifications have been characterized and may be represented by the categorical output 42. Non-limitative examples include N6-methyladenosine (m6A), Inosine (I), N6,2′-O-dimethyladenosine (m6Am), 8-oxo-7,8-dihydroguanosine (8-oxoG), pseudouridine (Ψ), 5-methylcytidine (m5C), and N₄-acetylcytidine (ac4C), have been shown to regulate mRNA stability and function.

Other types of embodiment are concerned with providing an estimate of the identity of one or more subject polymer units, for example to allow for detection of errors in a previously derived estimate of the sequence of polymer units and/or for detection of changes from a reference sequence. In this case, the output 42 represents an estimate of the identity of the subject polymer unit between categories including a set of canonical polymer units. For example in the case that the polymer unit is a DNA polynucleotide, the canonical nucleotides may be the four bases adenine (A), cytosine (C), guanine (G), and thymine (T).

This allows for detection of single nucleotide substitutions. When basecall anchoring is used, this is a corrective procedure aimed at improving a first pass prediction of the originating sequence. When reference anchoring is used, this represents the detection of single nucleotide polymorphisms (SNPs) wherein the provided reference sequence 23 does not match the originating sample via a single nucleotide substitution.

In addition to single nucleotide substitutions, it is possible for the categories to include small insertions or deletions (for example less than 50 nucleotides). A further category of modifications that can be detected using the algorithm is where a nucleotide has neither a purine nor a pyrimidine base, known as an abasic site. An abasic site may be generated due to for example, DNA damage, with depurination being more common. Depurination is thought to play a major role in the initiation of cancer. Abasic sites are routinely present in DNA but are also known to occur in the RNA of yeast and human cells.

In this case, the polymer unit prediction task may be adjusted to mask the subject polymer unit in the sequence slice 32 that is input to the slice machine learning system 41, so as not to bias the output predictions based on the input base.

In general, the slice machine learning system 41 may use a variety of different machine learning techniques. However, a particularly advantageous form the slice machine learning system 41 is as a neural network.

By way of illustration, FIG. 8 shows an example in which the slice machine learning system 41 is a neural network 50. There will now be described the features or components of the neural network 50 and training methods for such a neural network.

The neural network 50 comprises a first input stage 51 to which the sequence slice 31 is supplied, and a second input stage 52 to which the signal slice 32 is input.

The first input stage 51 comprises at least one first input neural network layer. The input neural network layer(s) of the first input stage 51 may be convolutional neural network layer(s).

The second input stage 52 also comprises at least one second input neural network layer. The input neural network layer(s) of the second input stage 52 may be convolutional neural network layer(s).

The outputs of the first and second input stages 51 and 52 are supplied to a concatenation layer 53 which concatenates those outputs to provide a concatenated output 54 that is supplied to the remaining layers, also comprising at least one convolutional neural network layer. The concatenation is performed feature-wise so that the temporal (sequencing signal time direction) correspondence between inputs to the concatenation layer 53 derived from the sequence slice 31 and the signal slice 32 is preserved. Output values from the concatenation layer 53 are then further processed by layers in the neural network 50 as a single input.

The further layers are arranged as follows.

The concatenated output 54 is supplied to a combined convolutional neural network stage 56 that comprises at least one convolutional neural network layer.

The convolutional neural network layers of the first and second input stages 51 and 52 and the combined convolutional neural network stage 56 may be of conventional construction. Such convolutional neural network layers are well known in the art, but in summary operates on fixed sized moving windows of the input data at a stride along the input data. At each window, the input features are matrix multiplied by a set of weights to produce the outputs of the layers.

Each of the first and second input stages 51 and 52 and the combined convolutional neural network stage 56 may include any number of convolutional layers stacked together, with different hyper-parameters being applied at each layer including window size, stride, and number of parameters/weights. Convolutional layers may each be followed by a batch normalization layer and an activation function (in this case swish nonlinearity) as well as other standard neural network components. The convolutional layers in the first and second input stages 51 and 52 are designed to produce the same output size in terms of the length and feature dimensions. Note that the input for each of the first and second input stages 51 and 52 has a different feature dimension size.

No padding is used with any of the convolutional layers as is common in some fields of machine learning when using convolutional layers.

The output of the combined convolutional neural network stage 56 is supplied to a LSTM (long short-term memory) stage 57 comprising at least one LSTM layer, which is an example of a recurrent neural network (RNN) layer, and may be of conventional construction.

The LSTM stage 57 is optional and may be omitted.

The output of the LSTM stage 57, or the output of the combined convolutional neural network stage 56 in the event that the LSTM stage is omitted, is supplied to a fully connected stage 58 comprising at least one fully connected layer, which again may be of conventional construction. The fully connected stage 58 produces the output 42.

A description of recurrent neural network layers that may be applied in the LSTM stage 57 and the fully connected stage 58 s given in Sak, H., Senior, A. W. and Beaufays, F., 2014. Long short-term memory recurrent neural network architectures for large scale acoustic modeling.

The neural network 50 processes the input in batches. The cross-entropy loss is calculated for each batch, as described above. An optimizer is used during training to backpropagate. In one demonstration the optimizer may be the AdamW optimizer.

Backpropagation is done in the standard fashion as described in prior art (Loshchilov, I. and Hutter, F., 2017. Decoupled weight decay regularization. arXiv preprint arXiv: 1711.05101).

Attention layers may also be added to the neural network 50 by calculating a ‘compatibility’ score between intermediate feature vectors and the global feature vector (the final output prior to activation). The intermediate features are found after the initial convolutions in each head of the network (signal and sequence) and post concatenation of these signals. The compatibility score can be in the form of the summation of the feature vectors with the global feature vector or in their dot product and the row-wise softmax is applied to turn these into attention vectors. These attention vectors are then used to create an element-wise weighted average of the intermediate feature vectors. These are then concatenated together and passed through a final layer as the classification step. The advantage of these layers is in allowing the attention maps to be visualized and helps understand which parts of the signal and/or sequence are being attended to make the prediction.

The neural network 50 may be trained using conventional techniques involving supply of the neural network with training signals comprising plural pairs of a training sequence slice 61 around a subject polymer unit in a sequence of polymer units of a polymer, and a training signal slice 62 of a measurement signal measured from the polymer during translocation of the polymer with respect to a nanopore, for example as shown in FIG. 9.

The training sequence slice 61 contains a subject polymers of known categories.

The training signal slice 62 is mapped to the training sequence slice 61. The input mapping 23 is derived using a consistent procedure as between the training and subsequent use of the trained neural network 50. When derived from a basecalling algorithm, the neural network 50 derives the nucleotide to this position. When derived from a k-mer or level model followed by dynamic programming the expected levels should represent the input polymer units. Both methods thus apply a consistent method with a meaningful sequence to signal mapping.

The training signals are prepared to provide examples of the categories of the desired output 42, as described above.

Where the categories represented by the output 42 are a canonical polymer unit and at least one modified form of the canonical polymer unit, then the training signals are annotated with known canonical and modified bases sequence. Like canonical substitution models, raw nanopore signal may be derived from any source biological material with a known reference or from which a genomic reference could be derived with high accuracy.

For a modified base model, the knowledge of the modified base content of reads may also have several sources.

For example, the source of ground truth modified bases may come from biological knowledge of a certain procedure or technique. As a specific example, bacterial methylase enzymes may be purchased from a supplier and used to treat a previously unmodified biological sample of known origin. This will generally convert nucleotides at a fixed sequence pattern (in sequence biology known as a motif) from the canonical form to a modified form. As a specific example, the M.SssI methyltransferase converts a canonical cytosine to 5-methyl-cytosine in any CG contexts. This biological process may be error prone. Biological or algorithmic methods may be developed to improve or filter this training reference modification markup.

Additional biological methods may also be applied to generate ground truth sets for further derived modifications from the procedure described above. For example, the Ten Eleven Translocase (TET) enzyme is known to catalyze an oxidation reaction to convert 5-methy-cytosine (5mC) to (in order of reaction mechanism) 5-hydroxymethyl-cytosine (5hmC), 5-formyl-cytosine (5fC) and 5-carboxyl-cytosine (5caC). Such samples may be processed by nanopore sequencing and used for training.

As another example of a type of the training signals, modified bases can be printed into oligonucleotides. These oligonucleotides can be ordered with fixed sequence with modified bases at known positions. The oligonucleotides can also be ordered with selected positions containing random bases. The identity of the random positions may be determined from the produced raw nanopore signal for that read or other aspects of the nanopore run (namely paired reads). These ground truth or partially random sequences are processed in the same manner as standard genomic reads to produce raw nanopore signal, ground truth sequence including modified base identities and a mapping between these two.

One final modified base training sample again starts from an unmodified reference sample. A polymerase chain reaction (PCR) is performed with this sample as the template input, with canonical nucleotides units (dNTPs) as well as a doped in rate of modified bases (e.g. d5mCTP or d5hmCTP). Given an acceptable polymerase which can accept such modified bases, modified nucleotides will incorporate into the daughter strands of a PCR reaction at random positions. The resulting sample will contain strands with known canonical sequence, but unknown modified base content. Such a sample would need to be marked up appropriately with a nanopore modified base detection model. This procedure may be error prone but may improve final model performance in future iterations of the model implemented in the slice machine learning system 41, especially if appropriate filtering or other algorithmic steps are applied.

Where the categories represented by the output 42 are a set of canonical polymer units, then the training signals are a set of reads with known canonical sequence. These training signals are identical to standard basecalling training, for example as applied to the initial machine learning system 11.

Raw nanopore signal for the training signals may be derived from any source biological material with a known reference sequence or from which a genomic/source reference sequence could be derived with high accuracy.

Nanopore reads are processed as described previously in with respect to reference-anchoring. This provides signal, ground truth sequence and a mapping between these two as input into the Remora algorithms. These are initially provided as whole nanopore read units and training/inference chunks are selected for each base of interest within a read as previously described.

The training may be performed using conventional techniques. The various layers of the neural network 50 describe above are connected, and weight matrices assigned to each later are designed such that matrix multiplication is performed with valid dimensions for the output and input of connected layers. Application of the neural network produces a vector of values representing the output categories of the prediction problem (modified baes or canonical substitution detection). A loss function is applied to this output layer along with a set of ground truth labels for each training unit. The most common loss function for multiclass prediction is cross entropy (as disclosed for example in Murphy, Kevin P. Machine Learning: A Probabilistic Perspective. MIT Press, 2012.) but others are available and applicable here. The training of the neural network 50 is performed to minimize the value of this loss function by iteratively updating the weights of all layers composing the neural network.

To minimize this loss value a batch of inputs are passed into the neural network 50 applying each layer as designed by the connections within the neural network 50. This produces a value from the loss function. An optimizer is then applied to this loss function. An optimizer observes the partial gradient of each parameter weight with the contribution to the loss value and propagates this difference backwards (from output back to the input) through the neural network. Weights are updated via a small fraction, according to the learning rate) of this difference. These updates move the neural network 50 in the direction of an improvement in the loss function value. This is the standard procedure for training a neural network.

Batching is applied to training signals in order use computing resources efficiently. Larger batches will generally produce more robust training, but also slow training due to increased compute requirements. A trade off these values is made given the computational resources available.

Other layers are applied only at training time to stabilize training. As an example, batch normalization layers may be added between any connection of other layers.

Non-linear activation functions (such as ReLU, Tanh, Sigmoid, swish and many others) may be applied between any connection between neural network layers as well (Sharma, Sagar, Simone Sharma, and Anidhya Athaiya. “Activation functions in neural networks.” towards data science 6.12 (2017): 310-316). Back propagation through such layers is defined by statistical principles and prior art.

A comparison was made between a specific embodiment of the method described above, referred to as the Remora algorithm, and some other prior art methods, as applied by way of example to the detection of 5-methyl-cytosine (5mC). In particular, the following methods were used for this comparison:
Tombo: v1.5.1 https://nanoporetech.github.io/tombo/
Deepsignal2: v0.1.1 https://github.com/PengNi/deepsignal2
f5c: v0.7 https://github.com/hasindu2008/f5c.
Guppy: 5.0.16 https://community.nanoporetech.com/downloads/guppy
Megalodon: v2.3.5 https://github.com/nanoporetech/megalodon
Present basecall as implemented in Remora software v0.1.0 https://github.com/nanoporetech/remora: An example of the method described above with basecall anchoring
Present reference as implemented in Remora software v0.1.0 https://github.com/nanoporetech/remora: An example of the method described above with reference anchoring

The Remora algorithm was trained using two enzymatically converted human genomic DNA samples. The first is treated with by polymerase chain reaction (PCR) to replace all bases with their canonical equivalent and the second is synthetically treated with the bacterial methylase M.Sss1 which converts all cytosines in a CG reference sequence context with 5mC.

Comparison of correlation coefficients between different nanopore signal tools and bisulfite sequencing (Darst, Russell P., et al. “Bisulfite sequencing of DNA.” Current protocols in molecular biology 91.1 (2010): 7-9.) for 5-methyl-cytosine detection aggregated at genomic position level is provided below to demonstrate the relative performance of the algorithms described here to the current prior art. The DNA material are extracted from the NA12878 reference human cell line sample (derived from the HG001 donor individual) (https://www.coriell.org/0/Sections/Search/Sample_Detail.aspx?Ref=NA12878).

Nanopore datasets were generated on ONT MinION flowcells (R9.4.1/E8) corresponding to a CsgG nanopore (R) and DdA enzyme (E) under standard conditions at a rate of translocation of approximately 450 bases/second and DNA samples were prepared for nanopore sequencing using the LSK109 library preparation kit, see for example https://store.nanoporetech.com/uk/ligation-sequencing-kit html and https://gib.og.edu.au/research/long-read-sequencing/beads-free-ont-ligation-kit-library-preparation-ultra-long-read-sequencing The coefficients were evaluated at different sequencing depth (average number of reads per genomic position), from 15 to 60. The results are shown in Table 1.

TABLE 1

| | Sequencing depth | | | | |
|---|---|---|---|---|---|
| Tools | 15 | 20 | 30 | 45 | 60 |
| Tombo | 0.893 | 0.889 | 0.883 | 0.88 | 0.878 |
| Deepsignal2 | 0.901 | 0.906 | 0.914 | 0.92 | 0.923 |
| f5c | 0.9 | 0.898 | 0.9 | 0.906 | 0.91 |
| Guppy | 0.887 | 0.891 | 0.902 | 0.912 | 0.916 |
| Megalodon | 0.906 | 0.908 | 0.915 | 0.922 | 0.925 |
| Remora | 0.903 | 0.905 | 0.913 | 0.92 | 0.924 |
| basecall | | | | | |
| Remora | 0.912 | 0.913 | 0.918 | 0.924 | 0.927 |
| reference | | | | | |

As shown in Table 1, from the same source data, the current algorithm (Remora) systematically outperforms the other known prior art algorithms in being able to detect 5-methyl-cytosine (5mC).

1. A method of analysing a measurement signal measured from a polymer during translocation of the polymer with respect to a nanopore, the polymer comprising a sequence of polymer units, the method comprising:
deriving an input sequence estimate of the sequence of polymer units, and a mapping between the measurement signal and the input sequence estimate,
supplying
a sequence slice derived from a slice of the input sequence estimate around a subject polymer unit in the sequence of polymer units, and
a signal slice of the measurement signal, the sequence slice and signal slice being mapped to each other by the mapping,
as inputs to a slice machine learning system that provides an output representing an estimate of the identity of the subject polymer unit.

2. A method according to claim 1, wherein the output represents an estimate of the identity of the subject polymer unit between categories including canonical polymer unit and at least one modified forms of the canonical polymer unit.

3. A method according to claim 2, wherein
the polynucleotide is DNA,
the polymer units are nucleotides,
the canonical polymer unit is cytosine or adenosine, and
the at least one modified form of the canonical polymer unit is at least one of 5-methyl-cytosine and 5-hydroxymethyl-cytosine in the case that the canonical polymer unit is cytosine or is 6-methyl-adenosine in the case that the canonical polymer unit is adenosine.

4. A method according to claim 1, wherein the output represents an estimate of the identity of the subject polymer unit between categories including a set of canonical polymer units.

5. A method according to any one of the preceding claims, wherein the method is performed for a subject polymer unit forming part of a predetermined motif comprising plural canonical polymer units.

6. A method according to any one of the preceding claims, wherein the method is performed in respect of plural subject polymer units in the sequence of polymer units.

7. A method according to any one of the preceding claims, wherein the step of deriving the input sequence estimate comprises supplying the measurement signal as an input to an initial machine learning system that provides an output that is an initial sequence estimate of the sequence of polymer units that is used as the input sequence estimate.

8. A method according to any one of claims 1 to 6, wherein
the input sequence estimate is a reference sequence in respect of the polymer,
the method comprises supplying the measurement signal as an input to an initial machine learning system that provides an output that is an initial sequence estimate of the sequence of polymer units, and
the step of deriving a mapping between the measurement signal and the input sequence estimate comprises:
deriving a reference mapping between the reference sequence and the initial sequence estimate, and a signal mapping between the measurement signal and the initial sequence estimate; and
deriving the mapping between the measurement signal and the input sequence estimate from the reference mapping and the signal mapping.

9. A method according to claim 7 or 8, wherein the initial machine learning system is arranged to provide a further output which is the mapping between the measurement signal and the initial sequence estimate.

10. A method according to claim 7 or 8, wherein the step of deriving the mapping between the measurement signal and the initial sequence estimate comprises:
generating a signal prediction of the signal predicted to be generated from the initial sequence estimate by a model of a measurement system used to provide the measurement signal, and
deriving the mapping by comparing the signal prediction with the measurement signal.

11. A method according to any one of the preceding claims, wherein the sequence slice is encoded as k-mers corresponding to respective polymer unit in the slice of the input sequence estimate, each k-mer comprising a group of k polymer units including the respective polymer unit and (k−1) adjacent polymer units from the input sequence estimate, where k is a plural integer.

12. A method of according to claim 11, wherein the k has a value in a range from 3 to 50.

13. A method according to claim 12, wherein k has a value selected so that the length of the k-mer is greater than the length of the nanopore lumen through which the polymer translocates.

14. A method according to any one of the preceding claims, wherein the signal slice is a predetermined length of the measurement signal around a position in the measurement signal mapped to the subject polymer unit.

15. A method according to any one of the preceding claims, wherein the sequence slice is expanded prior to supplying the sequence slice to the slice machine learning system such to have same size as the signal slice.

16. A method according to any one of the preceding claims, wherein the polymer units represented by the sequence slice are encoded in binary format prior to supplying the sequence slice to the slice machine learning system.

17. A method according to any one of the preceding claims, wherein the measurement signal is normalised prior to supplying the signal slice to the slice machine learning system.

18. A method according to any one of the preceding claims, wherein the slice machine learning system is a neural network.

19. A method according to claim 18, wherein
the slice machine learning system comprises at least one first input neural network layer to which the sequence slice is supplied, and at least one second input neural network layer to which the signal slice is supplied,
the slice machine learning system concatenates outputs of the at least one first convolutional neural network layer and the at least one second convolutional neural network layer, and
the slice machine learning system comprises further neural network layers to which the concatenated outputs are supplied as an input.

20. A method according to claim 19, wherein the at least one first input neural network layer and the at least one second input neural network layer are convolutional neural network layers.

21. A method according to claim 19 or 20, wherein the further neural network layers include at least one further convolutional neural network layer and/or at least one recurrent layer and/or at least one fully connected layer.

22. A method according to any one of the preceding claims, wherein the nanopore is a protein pore.

23. A method according to any one of the preceding claims, wherein the polymer is a polynucleotide, and the polymer units are nucleotides.

24. A method according to claim 23, wherein the polynucleotide is DNA.

25. A method according to claim 23 or 24, wherein the measurement signal is a measurement signal measured from a polymer during translocation of the polymer through a nanopore, wherein the rate of translocation of the polynucleotide through the nanopore is controlled by a molecular brake.

26. A method according to claim 25, wherein the molecular brake is an enzyme.

27. A method according to claim 26, wherein one or more nucleotides of the sequence slice are within a region of the enzyme that controls translocation of the polymer.

28. A method according to any one of the preceding claims, wherein the signal is derived from measurements of one or more of the following properties: ionic current, impedance, a tunnelling property, a field effect transistor voltage and an optical property.

29. A computer program comprising instructions which, when the program is executed by a computer, cause the computer to carry out a method according to any one of the preceding claims.

30. A computer storage medium storing a computer program according to claim 29.

31. A method of analysing a polymer comprising:
deriving a measurement signal from the polymer during translocation of the polymer with respect to a nanopore, the polymer comprising a sequence of polymer units; and
analysing the measurement signal using a method according to any one of claims 1 to 28.

32. An analysis apparatus comprising a processor configured to carry out a method according to any one of claims 1 to 28.

33. A nanopore measurement and analysis system comprising:
a measurement system arranged to derive a measurement signal from a polymer during translocation of the polymer with respect to a nanopore; and
an analysis apparatus according to claim 32.

34. A system according to claim 33 wherein the measurement system comprises a CsgG nanopore.

35. A system according to claim 33 or 34 wherein the binding enzyme is a helicase.

36. A method of training a slice machine learning system to provide an output representing an estimate of the identity of a subject polymer unit of interest in a polymer by supplying the machine learning system with training signals comprising plural pairs of
a training sequence slice around a subject polymer unit in a sequence of polymer units of a polymer, and
a training signal slice of a measurement signal measured from the polymer during translocation of the polymer with respect to a nanopore.
